(19) United States
(12) Patent Application Publication
(10) Pub. No.: US 2022/0088324 A1
(43) Pub. Date: Mar. 24, 2022

(54) PRESSURE VALVE FOR MEDICAL DEVICES AND METHODS OF USE

(71) Applicant: Boston Scientific Scimed, Inc., Maple Grove, MN (US)

(72) Inventors: Ryan EVERS, Billerica, MA (US); Daniel CONGDON, Hudson, MA (US); Andrew PIC, Northboro, MA (US); Collin MURRAY, Bolton, MA (US)

(73) Assignee: Boston Scientific Scimed, Inc., Maple Grove, MN (US)

(21) Appl. No.: 17/479,948

(22) Filed: Sep. 20, 2021

Related U.S. Application Data
(60) Provisional application No. 63/081,057, filed on Sep. 21, 2020.

(51) Int. Cl. A61M 13/00; F16K 15/02; F16K 27/02

(52) U.S. Cl. CPC A61M 13/00; A61M 2202/064; F16K 27/0209; F16K 15/025

(57) ABSTRACT

A medical device for delivering a material using a pressurized fluid includes an enclosure having a first chamber and a second chamber separated by a membrane, and an outlet, and a pin configured to move within the enclosure between a first position and a second position, where a proximal end of the pin blocks the outlet when the pin is in the first position.

### CROSS-REFERENCE TO RELATED APPLICATIONS

This application claims the benefit of priority of U.S. Provisional Patent Application No. 63/081,057, filed Sep. 21, 2020, the entirety of which is incorporated herein by reference.

### TECHNICAL FIELD

The disclosure relates generally to medical systems and devices for delivering pressurized fluids, and in examples, to methods and tools for controlling the release of a medical agent from a medical device using a pressurized fluid source.

### BACKGROUND

Fluid delivery systems and devices are used to supply various fluids, such as a gas, during medical procedures. These procedures may include supplying fluids within a range of appropriate pressures and/or flow rates. These fluids may include materials or agents, e.g., hemostatic agents, optimally delivered to tissue at an appropriate pressure and/or flow rate, for the particular application.

Medical fluid delivery systems often require delivering a fluid from a high pressure storage tank, such as a cartridge or similar housing, to a target via a housing containing a medical agent. Controlling the delivery of the medical agent may require appropriate dosing or metering of the medical agent to provide accurate and safe doses of the medical agent to the target. This may require controlling the release of the pressurized fluid and/or the medical agent. The disclosure may solve one or more of these problems or other problems in the art. The scope of the disclosure, however, is defined by the attached claims and not the ability to solve a specific problem.

### SUMMARY OF THE DISCLOSURE

According to an aspect, a medical device configured to deliver a material using a pressurized fluid includes an enclosure having a first chamber and a second chamber separated by a membrane, and an outlet, and a pin configured to move within the enclosure between a first position and a second position, wherein a proximal end of the pin is configured to block the outlet when the pin is in the first position.

The membrane may include a plurality of apertures fluidly connecting the first chamber and the second chamber.

The second chamber may be configured to contain the material, and wherein the membrane may be configured to prevent the material from moving from the second chamber to the first chamber.

The first chamber may include an inlet, and wherein the pressurized fluid may be configured to flow into the first chamber via the inlet.

The pin may be configured to move from the first position when a pressure of the pressurized fluid exceeds a pressure threshold.

The device may further include a spring, wherein the spring may be configured to urge the pin in the first position.

The pin may be configured to move from the first position when a pressure of the pressurized fluid overcomes a spring force exerted by the spring on the pin.

The pin may be configured to move from the first position when a pressure of the pressurized fluid overcomes a spring force exerted by the spring on the pin and a pressure of an exterior atmosphere exerted on the pin.

A distance between the proximal end of the pin and the membrane when the pin is in the second position may be sufficient to allow at least a portion of the material to move into the outlet from the second chamber.

The pin may be configured to move from the second position to the first position when the pressure of the pressurized fluid is below the pressure threshold.

The pin may include a protrusion, wherein the protrusion may be configured to extend into an opening of the outlet in the first position, to be disposed entirely outside the opening of the outlet in the second position, and to be disposed at least partially within the opening of the outlet in a third position of the pin between the first and second positions, wherein the pressurized fluid is flowable from the second chamber through the opening and the material is not flowable from the second chamber through the opening, when the pin is in the third position.

The pin may be configured to be moved to a third position, between the first position and the second position, when a pressure of the pressurized fluid in the first chamber is greater than a first threshold and lower than a second threshold.

The pressurized fluid may be configured to pass from the first chamber into the second chamber when the pin is in the third position, but the material may be configured to remain within the second chamber when the pin is in the third position.

The device may further comprise an actuator configured to supply the pressurized fluid to the first chamber when the actuator is actuated.

The device may further include a body having an input opening for receiving the pressurized fluid and an output opening for delivering the pressurized fluid, the body defining a fluid path between the input opening and the output opening, and a handle configured to receive an enclosure containing the pressurized fluid, wherein the enclosure is configured to be attached to the body and form a portion of the fluid path between the input opening and the output opening.

According to another aspect, a medical device configured to deliver a material includes a body having an input opening for receiving a pressurized fluid and an output opening for delivering the material, an enclosure having a first chamber and a second chamber, a membrane including a plurality of apertures, wherein the membrane is disposed between the first chamber and the second chamber, and a pin configured to move within the enclosure between a first position and a second position, wherein the pin is configured to close the output opening in the first position.

The second chamber may be configured to contain a material, and wherein a diameter of each of the plurality of apertures may be configured to be less than a particle size of particles of the material.

The pin may include a protrusion extending from a proximalmost end of the pin, and wherein the protrusion may be configured to extend into the output opening in the first position.

A distance between the protrusion and the output opening in the second position may be sufficient to allow a material and the propellant fluid to pass from the second chamber into the output opening.

According to another aspect, a method for controlling a material delivery to a body of a patient includes actuating an actuator to cause an enclosure containing a pressurized fluid to release the pressurized fluid from the enclosure, supplying the material to a first chamber, causing a pin in a second chamber, adjacent the first chamber, to move from a closed position to an open position, wherein the open position is configured to allow mixture of a material in the second chamber and the propellant fluid to pass from the second chamber, and supplying the mixture of the material in the second chamber and the propellant fluid to a target site.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a schematic of a delivery system according to an exemplary embodiment;

FIG. 2 is a cross-section of an enclosure of the delivery system of FIG. 1 according to an embodiment;

FIG. 3 is a cross-section of the enclosure of FIG. 2 according to an embodiment;

FIG. 4A is a cross-section of an enclosure of the delivery system of FIG. 1 according to another embodiment;

FIG. 4B is a cut away of an enclosure of the delivery system of FIG. 1 according to another embodiment; and

FIG. 5 is a cross-section of an enclosure of the delivery system of FIG. 1 according to another embodiment.

### DETAILED DESCRIPTION

The disclosure is described with reference to exemplary medical systems for dispensing a material or an agent (such as a therapeutic or hemostatic agent) using a pressurized fluid. The devices associated with the medical systems may improve the functionality and/or the safety of the medical systems by supplying the material in a regulated amount to a target site. In examples, an enclosure containing a material or an agent may be supplied with a pressurized fluid to agitate the material and supply the material in a proper dosed amount.

Reference to any particular procedure is provided in this disclosure only for convenience and not intended to limit the disclosure. A person of ordinary skill in the art would recognize that the concepts underlying the disclosed device and application method may be utilized in any suitable procedure, medical or otherwise. The disclosure may be understood with reference to the following description and the appended drawings, wherein like elements are referred to with the same reference numerals.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

For ease of description, portions of the device and/or its components are referred to as proximal and distal portions. It should be noted that the term “proximal” is intended to refer to portions closer to a user of the device or upstream in a propellant fluid path, and the term “distal” is used herein to refer to portions further away from the user or downstream in the propellant fluid path. Similarly, extends “distally” indicates that a component extends in a distal direction, and extends “proximally” indicates that a component extends in a proximal direction. Further, as used herein, the terms “about,” “approximately” and “substantially” indicate a range of values within +/−10% of a stated or implied value. Additionally, terms that indicate the geometric shape of a component/surface refer only to approximate shapes.

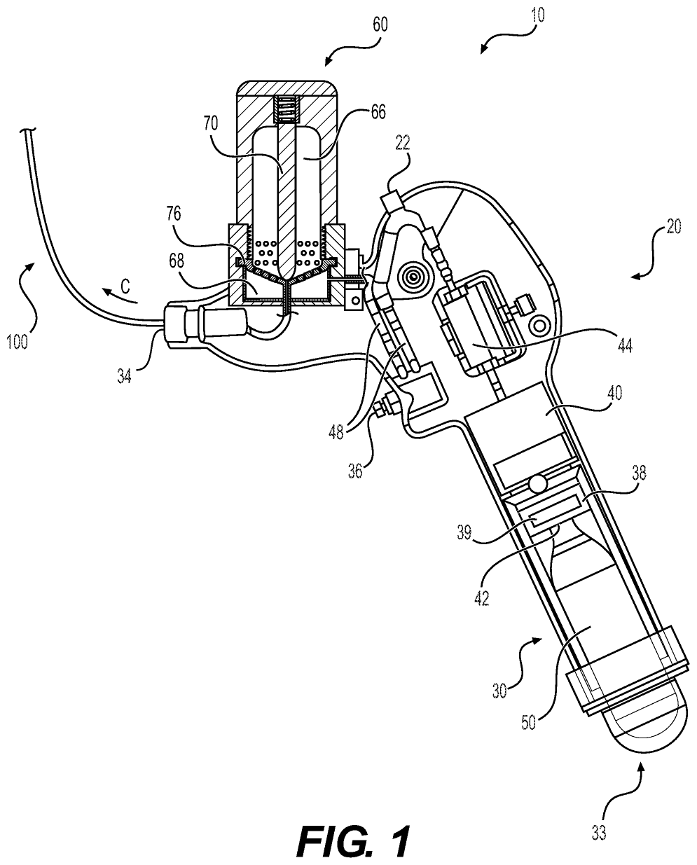

Referring to FIG. 1, a delivery system 10 according to an embodiment is shown. Delivery system 10 includes an application device 20, e.g., a hand-held device, having a handle 30 at a proximal end, and one or more triggers or actuators 36 configured to actuate delivery system 10 to release a propellant fluid. A tube 100 (e.g., a catheter), or an application tip, may be attached to a distal outlet 34 of delivery system 10 to aid in supplying the propellant fluid and/or a mixture of the propellant fluid and an agent (e.g., a hemostatic agent, a medical agent, or other agent) to a desired location. According to an example, outlet 34 may include a male or a female luer fitting, but is not limited to this configuration.

A containment device 50 (e.g., a cartridge or enclosure) may be contained within, or otherwise attached to, handle 30. Containment device 50 is configured to contain a propellant fluid, such as a gas, e.g., carbon dioxide or any other gas or fluid known in the art for dispensing material, such as a medical powder or reagent, into a patient at a target location. While shown as a torpedo-shape, containment device 50 may be any shape, such as a sphere, or any other shape known in the art for containing gas. For example, containment device 50 may be a carbon dioxide tank or cylinder typically found in medical settings, such as a hospital, and may be connected to application device 20 by a conduit (not shown). Containment device 50 includes one or more outer walls defining one or more inner chambers (not shown), the inner chamber(s) configured to contain the propellant fluid. The walls of containment device 50 may be formed of any material suitable for containing the propellant fluid, such as but not limited to a metal alloy, a ceramic, or other material known in the art. The propellant fluid contained in the inner chamber of containment device 50 may be under pressure. Accordingly, the walls are formed of a material and/or a thickness suitable to contain the propellant fluid at a pressure of, for example, approximately 1200 pounds per square inch (PSI), or approximately 850 PSI or less. For example, gases which may be contained in containment device 20 include carbon dioxide (CO2) having a vapor pressure of approximately 2,000-8,000 kPa at typical device temperatures, or nitrogen (N2) having a vapor pressure less than 40 MPa at typical device temperatures. It will be understood that these gases are examples and are not limiting to the types of gases contained in containment device 50.

With continued reference to FIG. 1, containment device 50 may be attached directly to application device 20 by any attachment device 38, including but not limited to screw-type connectors, pressure washer adapters, a pierce pin and seal arrangement, or any other device known in the art. It will also be understood that attachment device 38, or any other device for attaching containment device 50 to application device 20, may include an actuator 39 (e.g., a tab, a button, etc.) for opening or rupturing a burst disc or pressure release valve attached to containment device 50 and/or application device 20. Actuator 39 may be actuated at an end of a procedure to vent any remaining propellant fluid from containment device 50. An alert, such as a tactile or an audible alert, may be generated when containment device 20 is attached to application device 30. Alternatively, or additionally, actuator 39 may include a pierce pin to puncture a seal or membrane of an opening in containment device 50 to fluidly connect containment device 50 to application device 20.

In some cases, a cap 33 may be releasably attached to a proximalmost end of handle 30 and may control and/or assist in the attachment of containment device 50 to application device 20 within handle 30. In some instances, cap 33 may contact a proximalmost end of containment device 50 and may move or urge containment device 50 toward an inlet 32 of application device 20. Cap 33 may provide additional support to secure containment device 50 to application device 20. In some cases, cap 33 may be movably connected to handle 30 and may include a lever or similar handle (not shown) connected thereto. Movement of the lever may cause cap 33 to move in a proximal and a distal direction relative to handle 30. This movement may move containment device 50 toward and away from attachment device 38.

Once containment device 50 is attached to application device 20, actuation of one or more actuation devices 36 (only a single actuation device 36 is shown in FIG. 1) of application device 20 may cause the propellant fluid to be released from containment device 50, travel through application device 20, and release to catheter 100 through outlet 34 of application device 20. It will be understood that only one actuating device 36 may need to be actuated in some embodiments. Alternatively, or additionally, a plurality of actuating devices 36 may be simultaneously actuated to release propellant fluid as, for example, a safety precaution. In some cases, actuation of one or more actuating devices 36 may release a buildup of pressure within delivery system 10, causing a regulator 40 to release propellant fluid from containment device 50 at a predetermined pressure. Application device 20 may include a handle or grip, such as a garden-hose handle or other pistol-like configuration. Actuating device 36 may be any push button, trigger mechanism, or other device that, when actuated, opens a valve and releases propellant fluid.

As discussed above, one or more regulators 40 may assist in regulating an amount of propellant fluid released from containment device 20 at a specific pressure. For example, regulator 40 may be a dual stage regulator, or regulator 40 may be two single stage regulators, such as two piston regulators, aligned in series. Attachment device 38 may include a pierce pin to pierce a seal of containment device 50 when containment device 50 is attached to application device 20. A propellant fluid pressure may further be adjusted by a membrane regulator 44 provided in series after regulator 40. The combination of regulator 40 and membrane regulator 44 may reduce the pressure of gas from containment device 20 to an acceptable outlet pressure, i.e., a pressure of the gas and any material at outlet 34. A pressure of a gas within delivery system 10, after regulators 40, 44 in tubes 48 of application device 20, and at a target area in a patient, may be predetermined, based on the tissue to which the gas and material is being dispensed. Alternatively, or additionally, the pressure of the gas after regulators 40, 44 may be determined, at least in part, on a pressure necessary to open a valve in an enclosure storing an agent, as will be described herein. An acceptable pressure at outlet 34 may be approximately plus or minus 40% deviation from the target pressure, or approximately plus or minus 25% deviation from the target pressure. For example, regulator 40 may reduce inlet pressure of the dispensing propellant fluid to approximately 50-150 PSI, and membrane regulator 44 may subsequently reduce the propellant fluid to approximately 30-40 PSI. According to an example, regulator 40 and membrane regulator 44 reduce the propellant fluid to the desired output pressure of the propellant fluid based on a predetermined setting during manufacturing. Alternatively, or additionally, one or both of regulator 40 and membrane regulator 44 may include a mechanism (not shown) for adjusting the pressure of the propellant fluid output from each regulator. Further, the pressure of the propellant fluid at an outlet of membrane regulator 44 may be approximately equal to the pressure of the propellant fluid at outlet 34. Alternatively, the pressure of the propellant fluid at outlet 34 may be different from the pressure of the propellant fluid at the outlet of membrane regulator 44. In some cases, a burst or safety valve 22 may be in the fluid path of tubes 48, which may burst or release if a pressure of the propellant fluid downstream of regulators 40, 44 is greater the threshold. An example of delivery system 10, including one or more features described above, is shown in U.S. application Ser. No. 16/589,633, filed Oct. 1, 2019, the entirety of which is incorporated herein by reference.

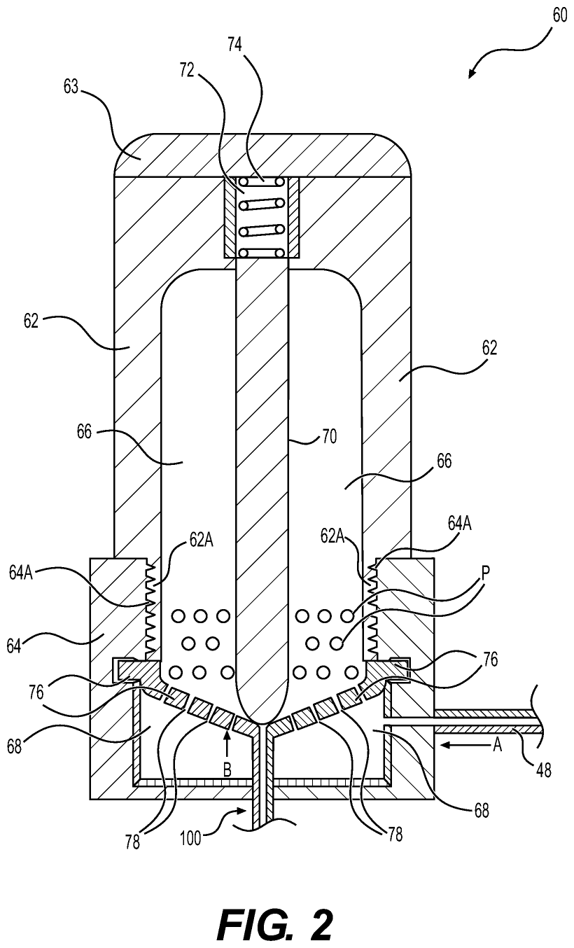

FIGS. 1 and 2 show an enclosure 60 configured to contain a material P (e.g., a powder or other medical agent, such as a hemostatic agent). Enclosure 60 is configured to store material P separate from the propellant fluid path absent a sufficient pressure exerted by the propellant fluid, as will be described herein. Alternatively, material P may be stored within the propellant fluid path, and an outlet of the fluid path may be closed or otherwise blocked off absent a sufficient pressure exerted by the propellant fluid.

As shown in FIG. 2, enclosure 60 may include a body 62 connected to a housing 64. Body 62 is shown as a cylindrical member, but may be any suitable shape. An outer diameter of at least a portion of an outermost wall of body 62 may be less than an inner diameter of at least a portion of an innermost wall of housing 64. In this manner, a portion of housing 64 may receive a portion of body 62 to connect body 62 to housing 64. In some examples, body 62 may be attached to housing 64 via screw threads 62a, 64a. Alternatively, body 62 may be connected to housing 64 via an adhesive, welding, or other technique known for sealing an enclosure to a medical device. A membrane 76, described herein, may be attached to body 62 and/or housing 64. For example, as shown in FIG. 2, a radially outermost portion of membrane 76 may sit or otherwise be attached (e.g., via an adhesive) within an annular slot in housing 64. Alternatively, or additionally, membrane 76 may be sandwiched between body 62 and housing 64. As will be described in greater detail, membrane 76 may define a barrier between chambers 66, 68.

With continued reference to FIG. 2, chamber 66 is defined by an outer wall of body 62 on three sides (e.g., a top surface and side surfaces in FIG. 2) and membrane 76 at a bottom surface of chamber 66 in FIG. 2. While chamber 66 is shown as being generally cylindrical, chamber 66 may take any shape suitable for containing and dispensing of material P. A volume of chamber 66 may be approximately 2 inches³ to 8.5 inches³, but is not limited thereto. For example, a volume of chamber 66 may increase or decrease based on, e.g., an amount of material P, the size of particles of material P, the type of medical procedure to be performed using delivery system 10, and/or other factors.

A pin 70 may extend into and may be movable relative to chamber 66. A lumen 72 is defined through a top wall of body 62 in FIG. 2. Lumen 72 may receive a portion of pin 70 as pin 70 moves relative to chamber 66. A diameter of an outermost surface of pin 70 may be approximately equal to a diameter of an innermost wall defining lumen 72. Additionally, or alternatively, a seal (not shown), such as a rubber sealing ring or the like, may be positioned at a proximal end of lumen 72 (a bottom area of lumen 72 in FIG. 2) to seal off lumen 72 from chamber 66. This seal may prevent propellant fluid and/or material P from entering lumen 72 during use.

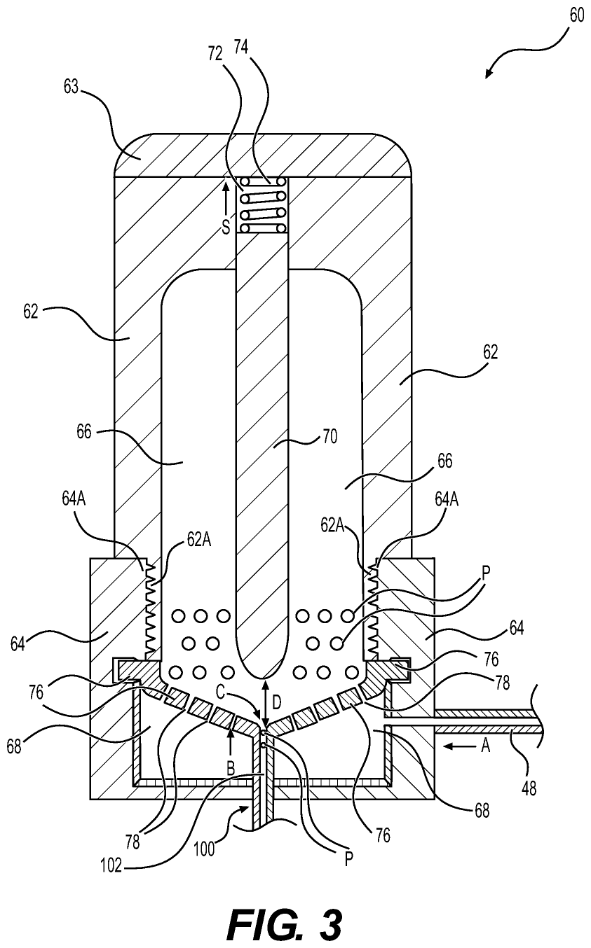

A cap 63 may cover a topmost portion of body 62, as shown in FIG. 2. Spring 74 may extend from and may be attached to cap 63 at a first end, may extend into lumen 72, and may be attached to a topmost facing surface of pin 70 via a second end, opposite the first end. As will be described herein, spring 74 may bias pin 70 toward membrane 76 in a closed position (e.g. a first position), opposite the direction of arrow S in FIG. 3. Additionally, or alternatively, lumen 72 may be exposed to an exterior atmosphere to assist pin 70 to achieve the closed position when the propellant gas is not flowing, as will be described herein. For example, pin 70 may be biased in the closed position by a combination of the spring force from spring 74 and the pressure of the exterior atmosphere.

With reference to FIGS. 2 and 3, a bottom end of pin 70 (an end opposite the end connected to spring 74) may be rounded or tapered. The tapered end of pin 70 may seal a proximalmost opening of a lumen 102 of tube 100 when pin 70 is in the closed position. In other cases, some (or all) of apertures 78 are uncovered or only partially covered by pin 70. The opening in lumen 102 may be an outlet of chamber 66. For example, as shown in FIG. 2, the tapered end of pin 70 may sit against a surface of membrane 76 facing chamber 66 so as to seal the proximalmost end of lumen 102 (e.g., a pathway exiting chamber 66). When the propellant fluid is at a sufficient pressure to overcome the force of spring 74 and/or the pressure of the exterior atmosphere, pin 70 may move in the direction of arrow S, shown in FIG. 3, to expose the proximalmost opening of lumen 102, as will be explained herein.

With reference to FIG. 2, membrane 76 may be Y-shaped, funnel-shaped, or cone-shaped, with the walls of membrane 76 tapering from sidewalls of body 64 away from chamber 66. According to an example, at least a portion of membrane 76 may have a shape similar to the tapered shape of pin 70. Membrane 76 may include a plurality of apertures 78 fluidly connecting chamber 66 to chamber 68. A diameter of apertures 78 may be large enough to allow a fluid gas (e.g., the propellant fluid) to pass from chamber 68 into chamber 66 in a direction indicated by arrow B, but may be small enough to prevent material P from passing from chamber 66 into chamber 68 via apertures 78. In some examples, apertures 78 may include a lattice structure forming random paths from a first side of membrane 76 to a second side, opposite the first side. A diameter of apertures 78 may be approximately 0.0015 inches to 0.004 inches. The diameter of apertures 78 may be larger than 100 microns, however. Further, each aperture 78 may have a same diameter from the first side of membrane 76 to the second side, or the diameter of some apertures 78 may change from the first side of membrane 76 to the second side. It will be understood that a diameter of apertures 78 may be selected based on the size of the particles of material P to be housed in chamber 68. For example, a diameter of apertures 78 may be larger if the size of the particles of material P is larger, and vice versa. In addition, the shapes and sizes of the apertures may be uniform, and the distribution of apertures about the membrane, or the sizes, shapes, and distribution may vary, as appropriate.

With continued reference to FIGS. 2 and 3, chamber 68 is formed between a proximalmost surface of membrane 76 (the bottom surface of membrane 76 in FIGS. 2 and 3) and housing 64. A tube 48 allows fluid to pass from regulators 40, 44 (downstream in the fluid path of containment device 50) to chamber 68 when one or more actuation devices 36 is actuated. For example, the fluid passes from containment device 50 into chamber 68 via regulators 40, 44 tube 48 in the direction indicated by arrow A.

When the propellant fluid is not activated and is not flowing into chamber 68, or when the propellant fluid flowing into chamber 68 is below a threshold, such as a pressure sufficient to overcome the spring force of spring 74 and/or the pressure of the exterior atmosphere, pin 70 remains in the closed position (FIG. 2). However, once the pressure of propellant fluid is sufficient to overcome the spring force of spring 72 and/or the pressure of the exterior atmosphere, the propellant fluid pushes against the tapered portion of pin 70 and forces pin 70 in the direction (indicated by arrow S in FIG. 3) and into an open position (e.g., a second position). In the open position, a distance D is defined between a proximalmost facing surface of the tapered surface of pin 70 and a distalmost facing surface of membrane 70. Distance D may be a distance suitable for a mixture of propellant fluid and material P to flow from chamber 66 to a target site via lumen 102 of tube 100. For example, distance D may be approximately 0.0625 inches to approximately 0.25 inches. It will be understood, however, that the distance D may be selected to be large enough to allow the mixture to flow unimpeded from chamber 66, through the outlet in chamber 66, and into the proximalmost end of lumen 102, as shown by arrow C in FIG. 3. In some cases, pin 70 may be in a third position, between the first position and the second position. The third position may cause a space to form between pin 70 and membrane 76, but may be insufficient to allow material P to pass into lumen 102. In this case, material P may be agitated by the propellant fluid prior to supplying the mixture to the target site. Pin 70 may be moved to the third position by the pressure of the propellant gas being above a first threshold and below a second threshold (e.g., the first threshold may be sufficient to begin to move spring 74, and the second threshold may be sufficient to overcome an additional amount or the entire spring force of spring 74).

A method of applying an agent (e.g., a medical agent or a hemostatic agent) to a target site using medical system 10 will be described with reference to FIGS. 1-3. Containment device 50 is attached to handle 30 via, e.g., attachment device 38. In one example, containment device 50 is inserted into a lumen of handle 30 and containment device 50 is moved toward inlet 30 via, e.g., cap 33. In another example, a tube may be used to connect containment device 50 (such as a hospital line having a propellant gas) to inlet 32. At this time, pin 70 is urged against membrane 76 and the proximalmost opening of lumen 102, as shown in FIG. 2.

Once the propellant gas supply is fluidly connected to application device 20, a user may activate medical system 10 by actuating one or more actuation devices 36. Actuation of the one or more actuation devices 36 causes propellant fluid to flow from containment device 50, through regulators 40, 44 and along tube 48, and into chamber 68, as shown by arrow A in FIG. 2. As the propellant fluid fills chamber 68, the pressure of the propellant fluid flowing into chamber 68 and through apertures 78 may cause pin 70 and spring 74 to move in a direction indicated by arrow S (FIG. 3). For example, spring 74 may be selected to have a spring force less than a pressure force of a pressurized fluid passing through regulators 40, 44. That is, regulators 40, 44 may allow propellant fluid to enter chamber 68 with a pressure sufficient to overcome the spring force of spring 74. In this manner, pin 70 is moved in the direction indicated by arrow S.

In some cases, the proximalmost end of pin 70 covers every aperture 78. In other cases, some (or all) of apertures 78 are uncovered or only partially covered by pin 70. As the propellant gas passes into chamber 66 via apertures 78 (in the direction indicated by arrow B), the propellant fluid mixes with material P. In some cases, the propellant fluid may mix with material P before pin 70 is moved from the closed position to the open position. Once pin 70 is in the open position, as shown in FIG. 3, the mixture of propellant fluid and material P may be supplied to the target site via lumen 102. When a user deactivates actuation device 36, pin 70 moves in a direction opposite arrow S and returns to the closed position (FIG. 2). Actuating actuation device 36 again (e.g., a second time) may cause the propellant fluid to flow into chamber 68 and cause pin 70 to move to the open position of FIG. 3. In this manner, a user may selectively supply material P to the target tissue in a controlled manner.

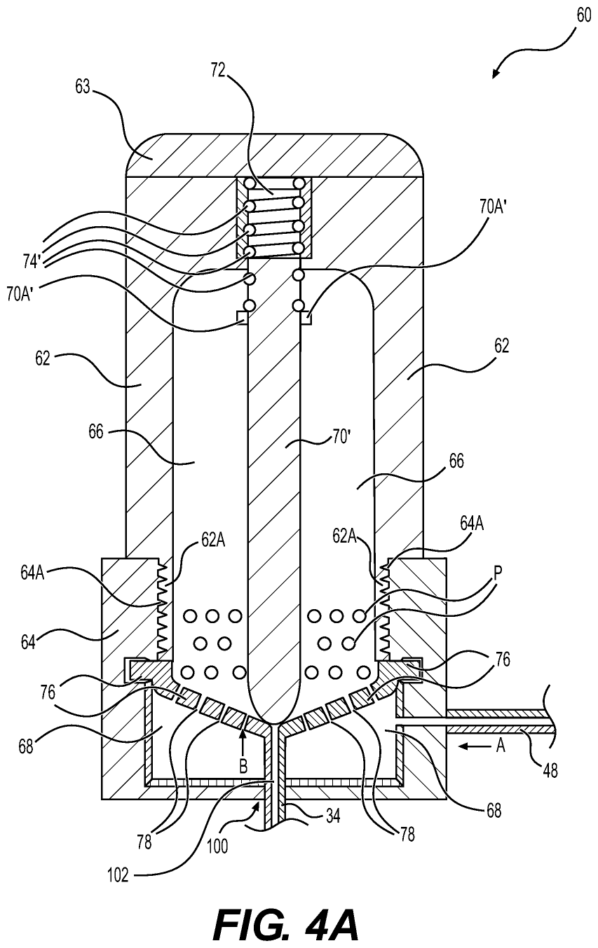

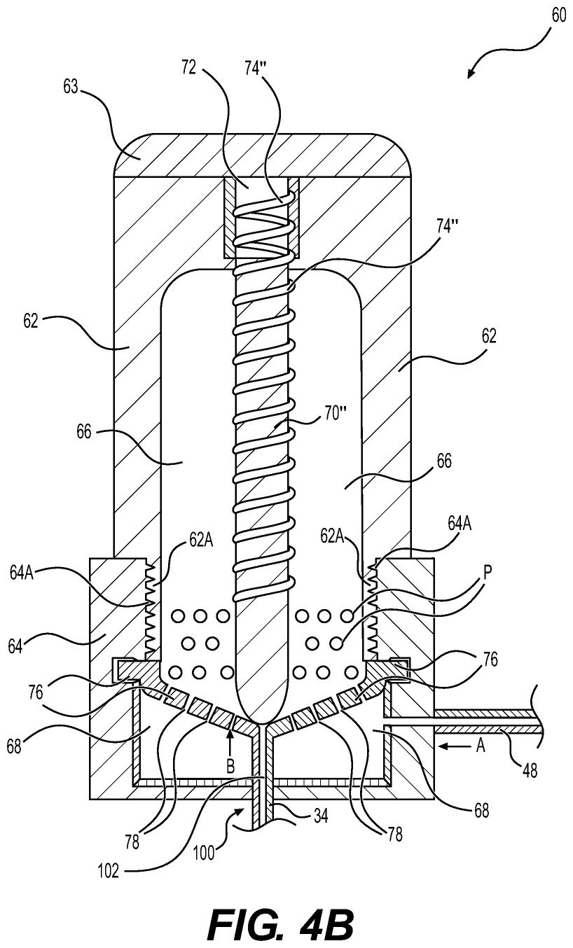

With reference to FIGS. 4A and 4B, two alternative embodiments for returning a pin 70′, 70″ to a closed position are shown. In FIG. 4A, spring 74′ may be integrally formed with housing 62. For example, a portion of spring 74′ may be partially embedded in housing 62, e.g., during molding of housing 62. In this example, spring 74′ may surround a circumference of at least a portion of pin 70′. Pin 70′ may include a protrusion 70a′ to connect spring 74′ to pin 70′. Protrusion 70a′ may be an annular ring, or protrusion 70a′ may include one or more protrusions about a circumference of pin 70′ to which spring 74′ may connect. In this case, spring 74′ may not move, and pin 70′ may move relative to lumen 72′.

With reference to FIG. 4B, a spring 74″ is disposed within lumen 72″ of housing 62. In this example, spring 74″ may not be formed with housing 62. Rather, spring 74″ may be connected to body 62 and/or cap 63 at a first end, and may be attached to pin 70″ at one or more points along spring 74″. Spring 74″ surrounds a portion of pin 70″. In both FIGS. 4A and 4B, a seal, such as an annular seal, may be disposed at a proximal end of lumens 72′, 72″ to seal off and prevent propellant fluid and/or material P from entering lumens 72′, 72″.

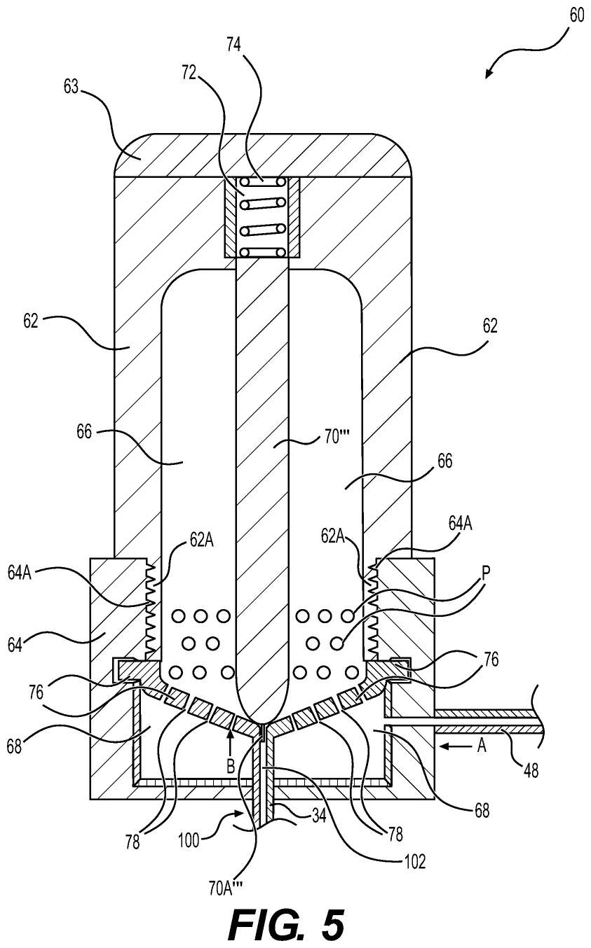

With reference to FIG. 5, a pin 70′″ according to another example is shown with a protrusion 70A′″ extending in a proximal direction from a proximalmost tip of pin 70′″. Protrusion 70A′″ may extend into lumen 102 of tube 100. A diameter of a radially outermost surface of protrusion 70A′″ may be less than a diameter of a radially innermost surface defining lumen 102, which may allow protrusion 70A′″ to move relative to lumen 102. As pin 70′″ moves in the direction indicated by arrow S, protrusion 70A′″ may slide out of lumen 102, such that a portion of protrusion 70A′″ is disposed within chamber 66. The outer diameter of protrusion 70A′″ and the inner diameter of lumen 102 are such to allow an annular space between protrusion 70A′″ and the walls of lumen 102. This annular space is large enough to permit ingress of the pressurized fluid, but small enough to not allow material P into lumen 102. In some instances, the outer diameter of protrusion 70A′″ may be approximately 0.005 inches to 0.050 inches. While protrusion 70A′″ is shown as a cylindrical shape, the shape is not limited thereto, and may be a cone, parabolic reducing to a point, or oval in shape. In some examples, the inner diameter of lumen 102 may be approximately 0.050 inches to 0.125 inches. For example, as propellant fluid is released into chamber 68, the pressure of the propellant fluid may be insufficient to overcome the spring force of spring 74′″ to move pin 70′″ from the closed position (e.g., FIG. 2) into a completely open position (e.g., FIG. 3). Until the pressure of the propellant fluid in chamber 68 is sufficient to completely overcome the spring force of spring 74, at least a portion of protrusion 70A′″ may remain within lumen 102. This may be beneficial to assist in purging lumen 102, e.g., to remove any material trapped with lumen 102, without also supplying material P from chamber 66 to lumen 102. Additionally, or alternatively, the user may mix or agitate material P within chamber 66 prior to releasing the propellant fluid and material P mixture. For example, the one or more actuators 36 may include one or more settings to release the propellant fluid at a specified pressure. In some instances, the one or more actuators 36 may be actuated to release the propellant fluid at a pressure sufficient to purge lumen 102 and/or mix material P in chamber 66. In other instances, the one or more actuators 36 may be actuated to release the propellant fluid at a pressure sufficient to overcome the spring force of spring 74′″ and move pin 70′″ into the completely open position, allowing the mixture of the propellant fluid and material P to travel into lumen 102 (e.g., as shown by arrow C in FIG. 3). For example, the one or more actuators 36 may be depressed to a first position to release the propellant fluid at a first pressure range, and to a second position to release the propellant fluid at a second position to release the propellant fluid at a second pressure range, different from the first pressure range. Alternatively, or additionally, a first actuator 36 may release the propellant fluid at the first pressure range, and a second actuator 36 may release the propellant fluid at the second pressure range.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device configured to deliver a material using a pressurized fluid, the device comprising:
an enclosure having a first chamber and a second chamber separated by a membrane, and an outlet; and
a pin configured to move within the enclosure between a first position and a second position,
wherein a proximal end of the pin is configured to block the outlet when the pin is in the first position.

2. The medical device according to claim 1, wherein the membrane includes a plurality of apertures fluidly connecting the first chamber and the second chamber.

3. The medical device according to claim 1, wherein the second chamber is configured to contain the material, and wherein the membrane is configured to prevent the material from moving from the second chamber to the first chamber.

4. The medical device according to claim 1, wherein the first chamber includes an inlet, and wherein the pressurized fluid is configured to flow into the first chamber via the inlet.

5. The medical device according to claim 1, wherein the pin is configured to move from the first position when a pressure of the pressurized fluid exceeds a pressure threshold.

6. The medical device according to claim 1, further comprising a spring, wherein the spring is configured to urge the pin in the first position.

7. The medical device according to claim 6, wherein the pin is configured to move from the first position when a pressure of the pressurized fluid overcomes a spring force exerted by the spring on the pin.

8. The medical device according to claim 6, wherein the pin is configured to move from the first position when a pressure of the pressurized fluid overcomes a spring force exerted by the spring on the pin and a pressure of an exterior atmosphere exerted on the pin.

9. The medical device according to claim 1, wherein a distance between the proximal end of the pin and the membrane when the pin is in the second position is sufficient to allow at least a portion of the material to move into the outlet from the second chamber.

10. The medical device according to claim 1, wherein the pin is configured to move from the second position to the first position when the pressure of the pressurized fluid is below the pressure threshold.

11. The medical device according to claim 1, wherein the pin includes a protrusion, wherein the protrusion is configured to extend into an opening of the outlet in the first position, to be disposed entirely outside the opening of the outlet in the second position, and to be disposed at least partially within the opening of the outlet in a third position of the pin between the first and second positions, wherein the pressurized fluid is flowable from the second chamber through the opening and the material is prevented from flowing from the second chamber through the opening, when the pin is in the third position.

12. The medical device according to claim 1, wherein the pin is configured to be moved to a third position, between the first position and the second position, when a pressure of the pressurized fluid in the first chamber is greater than a first threshold and lower than a second threshold.

13. The medical device according to claim 12, wherein the pressurized fluid is configured to pass from the first chamber into the second chamber when the pin is in the third position, but the material is configured to remain within the second chamber when the pin is in the third position.

14. The medical device according to claim 1, further comprising an actuator configured to supply the pressurized fluid to the first chamber when the actuator is actuated.

15. The medical device according to claim 1, further comprising:
a body having an input opening for receiving the pressurized fluid and an output opening for delivering the pressurized fluid, the body defining a fluid path between the input opening and the output opening; and
a handle configured to receive an enclosure containing the pressurized fluid,
wherein the enclosure is configured to be attached to the body and form a portion of the fluid path between the input opening and the output opening.

16. A medical device configured to deliver a material, the device comprising:
a body having an input opening for receiving a pressurized fluid and an output opening for delivering the material;
an enclosure having a first chamber and a second chamber;
a membrane including a plurality of apertures, wherein the membrane is disposed between the first chamber and the second chamber; and
a pin configured to move within the enclosure between a first position and a second position, wherein the pin is configured to close the output opening in the first position.

17. The medical device according to claim 16, wherein the second chamber is configured to contain a material, and wherein a diameter of each of the plurality of apertures is configured to be less than a particle size of particles of the material.

18. The medical device according to claim 16, wherein the pin includes a protrusion extending from a proximalmost end of the pin, and wherein the protrusion is configured to extend into the output opening in the first position.

19. The medical device according to claim 18, wherein a distance between the protrusion and the output opening in the second position is sufficient to allow a material and the propellant fluid to pass from the second chamber into the output opening.

20. A method for controlling a delivery of material delivery to a body of a patient, the method comprising:
actuating an actuator to cause an enclosure containing a pressurized fluid to release the pressurized fluid from the enclosure;
supplying the material to a first chamber;
causing a pin in a second chamber, adjacent the first chamber, to move from a closed position to an open position, wherein the open position is configured to allow mixture of a material in the second chamber and the propellant fluid to pass from the second chamber; and
supplying the mixture of the material in the second chamber and the propellant fluid to a target site.
